(19) United States
(12) Patent Application Publication
(10) Pub. No.: US 2025/0195523 A1
(43) Pub. Date: Jun. 19, 2025

(54) ORGANIC MOLECULES FOR TREATING MYELIN PATHOLOGIES

(71) Applicants: ICM (INSTITUT DU CERVEAU ET DE LA MOELLE ÉPINIÈRE), Paris (FR); INSTITUT NATIONAL DE LA SANTÉ ET DE LA RECHERCHE MÉDICALE, Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR); UNIVERSITE CLAUDE BERNARD LYON 1 (UCBL), Villeurbanne Cedex (FR)

(72) Inventors: Jean-Baptiste HURE, Mantes La Ville (FR); Carlos PARRAS, Paris (FR); Olivier RAINETEAU, Bron (FR); Louis FOUCAULT, Lyon (FR)

(73) Assignees: ICM (INSTITUT DU CERVEAU ET DE LA MOELLE ÉPINIÈRE), Paris (FR); INSTITUT NATIONAL DE LA SANTÉ ET DE LA RECHERCHE MÉDICALE, Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR); UNIVERSITE CLAUDE BERNARD LYON 1 (UCBL), Villeurbanne Cedex (FR)

(21) Appl. No.: 18/849,056

(22) PCT Filed: Mar. 23, 2023
(86) PCT No.: PCT/EP2023/057534

§ 371 Date: Sep. 20, 2024

(30) Foreign Application Priority Data
Mar. 23, 2022 (EP) 22305345.5

(51) Int. Cl. A61K 31/519; A61K 31/4453; A61P 25/28

(52) U.S. Cl. CPC A61K 31/519; A61K 31/4453; A61P 25/28

(57) ABSTRACT

The use of small organic molecules that genes involved in the calcium signal pathway for methods of treating or preventing myelin pathologies, such as, for example, multiple sclerosis and myelin pathologies associated with preterm birth. The methods involve administering to a subject in need thereof the small organic molecules or pharmaceutical compositions including the small organic molecules.

### FIELD

The present invention relates to the use of small organic molecules for treating or preventing myelin pathologies, such as, for example, multiple sclerosis and myelin pathologies associated with preterm birth.

### BACKGROUND

Perinatal hypoxia and neuroinflammation are two key factors affecting oligodendrocyte (OL) development, survival and myelination. For instance, oligodendroglial development is particularly affected in the context of early preterm birth (PTB), leading to hypomyelination, abnormal connectivity, and synaptopathy. PTB is the commonest cause of death and disability in children under 5 years, affecting 15 million infants yearly born before 37 gestational weeks (GW). Rates are increasing in developing countries (7% in France and UK, 13% in the US) but over the last decade, medical, pharmacological and technological advances have enhanced the survival rate of younger infants. Because of associated increased morbidity, PTB is an important public health concern, with many very low birth weight (<1.5 Kg) surviving children developing neonatal encephalopathy and white matter abnormalities leading to cognitive, behavioral, and attention deficits from into adulthood (such as autism spectrum, epilepsy).

Another pathology threatening OL development is multiple sclerosis (MS) that is a chronic autoimmune-mediated disease characterized by focal demyelinated lesions of the central nervous system (CNS). Although current immunomodulatory therapies can reduce the frequency and severity of relapses, they show limited impact on the progression of disease and do not solve the demyelination process.

Recent advances have identified several approaches to stop the immune attack and neuroinflammation towards OLs. However, so far, no medication has been approved to promote remyelination in the context of PTB or MS. Because of the long-term consequences and the absence of current treatments inducing remyelination, there is an urgent need to develop novel therapeutic approaches for promoting brain repair through OL regeneration.

In the present invention, the inventors have surprisingly discovered that small organic molecules, such as, for example, dyclonine hydrochloride and calcium folinate, have a strong pro-oligodendrogenic activity and promote remyelination in the context of PTB and MS.

### SUMMARY

The present invention relates to a small organic molecule that targets the genes involved in the calcium signaling pathway for use in treating or preventing a myelin pathology in a subject in need thereof.

In one embodiment, said small organic molecule crosses the blood-brain barrier.

In one embodiment, said small organic molecule is selected from the group comprising or consisting of folinic acid, dyclonine, or salts thereof, and any combination thereof.

In one embodiment, said small organic molecule is folinic acid or a salt thereof, preferably the small organic molecule is calcium folinate.

In one embodiment, said small organic molecule is dyclonine or a salt thereof, preferably the small organic molecule is dyclonine hydrochloride.

In one embodiment, the myelin pathology is multiple sclerosis.

In one embodiment, the myelin pathology is associated with preterm birth.

In one embodiment, the myelin pathology is associated with perinatal hypoxia.

In one embodiment, said small organic molecule is to be administered orally, by injection, topically, nasally, by inhalation, buccally, rectally, intratracheally, by endoscopy, transmucosally, by percutaneous administration. In one embodiment, said small organic molecule is to be administered orally or nasally.

In one embodiment, said small organic molecule is to be administered at a dose ranging from about 0.01 to about 100 μg per gram per intake (i.e. from about 0.01 to about 100 mg per kg per intake), preferably from about 0.1 to about 10 μg per gram per intake (i.e. from about 0.1 to about 10 mg per kg per intake), more preferably from about 0.5 to about 5 μg per gram per intake (i.e. from about 0.5 to about 5 mg per kg per intake). In one embodiment, said small organic molecule is to be administered daily.

In one embodiment, said subject has received, is receiving or will receive an anti-inflammatory agent. In one embodiment, said anti-inflammatory agent is selected from the group comprising or consisting of teriflunomide, fingolimod and dimethyl fumarate.

The present invention further relates to a pharmaceutical composition for use in treating or preventing a myelin pathology in a subject in need thereof comprising, consisting of or consisting essentially of the small organic molecule as described hereinabove and a pharmaceutically acceptable excipient.

### BRIEF DESCRIPTION OF THE DRAWINGS

FIGS. 1 (A-C) and 1(D-G) are a combination of schemas and histograms showing the oligodendrogenic activity of small molecules in neonatal neural progenitor cultures. A: Schematic representation of the protocol of neurosphere-derived neural progenitor cell cultures and drug administration. B: Histogram showing the ratio of oligodendroglial cells (PDGFRα⁺ OPCs and CNP⁺ OLs) (fold-change compared to vehicle) for each drug (i.e. SM1 to SM11) at a concentration of 750 nM. C-D: Histograms showing respectively the ratio of astrocytes GFAP+ cells (C), neuronal β-III-tubulin+ cells (D) as fold-change compared to vehicle for each drug condition at 750 nM. E-F: Histograms showing respectively the ratio of cell density (E) and differentiation (marker+ cells, (F) as fold-change compared to vehicles for each drug condition at 750 nM. G: Histogram showing the quantification of iOLs (Sox10^{high} cells) presented as fold-change (FC) between drug treatment and vehicle condition. Data are presented as mean+/−SEM of fold change normalized to vehicle. N=5 (B-F) and 6 (G) independent experiments. *p<0.05; **p<0.01; ***p<0.001. Statistical linear mixed-effects models followed by Type II Wald chi-square tests ANOVA.

FIG. 2 (A-B) is a combination of schemas and histograms showing the oligodendrogenic activity of small molecules in primary OPC cultures. A: Schematic representation of the protocol of purified OPCs by MACSorting with PDGFRα antibodies cultures and drug administration. B: Violin plots quantifying the cell surface complexity of Plp-GFP+OLs (μm2) and showing increased complexity in treated conditions. Data are presented as mean+/−SEM from 3 independent experiments. **p<0.01; ****p<0.0001. ANOVA with Dunnett's post hoc test. DMSO, vehicle for most molecules; CT, vehicle for Sm11.

FIG. 3 (A-C) is a combination of schemas and histograms showing the pro-oligodendrogenic effect of small molecules in ex-vivo cerebellar culture. A: Schematics illustrating the protocol of the cerebellar explant culture model from postnatal day 0 (P0) cerebellum. B-C: Histogram representing the differentiation index (SOX10+CC1+/SOX10+ cells) (B) and the Myelination index (surface MBP+CaBP+/surface CaBP+ (C)). Data are presented as mean+/−SEM of fold change normalized to vehicle. N=5 independent experiments (1-3 confocal acquisitions for each cerebellum). *p<0.05; **p<0.01; ***p<0.001. Statistical unpaired bilateral Wilcoxon Mann Whitney test.

FIGS. 4 (A-D), 4(E-F) and 4 (G-I) are a combination of histograms showing that the oral administration of sm5 and sm11 increases oligodendroglial number, OPC proliferation and differentiation in the focal white matter demyelination model and reduces the microglial activity. A: Monitoring of the daily oral drink water consumption (volumes in mL per day per mice). B-E: Quantification (Olig2+ cells per mm²) of the oligodendroglial cell density (B), density of OPCs (Olig2^{high}/Olig1^{high}) (C), density of proliferative OPCs (Mcm2+ PDGFRα+ cells or Ki67+/PDGFRα+ cells) (D) and percent of proliferating OPCs (E). F: Ratio of differentiating OLs (iOL2s, (Olig2^{high}/CC1^{high}/Olig1^{high})) per OPCs. G-H: Histograms depicturing in the lesion site the percent of Iba1+ cells (G) and Cd68+ cells (H). I: Histogram representing the density of pro-regenerative microglia (Arg1+ area). Data are presented as Mean±SEM. *p<0.05; **p<0.01; ***p<0.001; ****p<0.0001. One-way ANOVA statistical test

FIGS. 5 (A-C) and 5D are a combination of schemas and histograms showing that the intranasal administration of sm5 and sm11 rescues OL differentiation in white matter after neonatal hypoxia. A: Schematics protocol for the induction of neonatal hypoxia. Mice are under hypoxic chamber for 8 days (10% O2) or normoxia before 3 days of intranasal small molecules administration (control dPBS). EdU administration was done 30 mn before sacrifice (P19). B: Histogram depicturing the oligodendrocytes lineage cells density in cortical area. C: Histogram depicturing the differentiation ratio (Olig2+ CC1+/Olig2+). Normoxia N=5; Hypoxia N=5; Hypoxia+Sm5 N=4; Hypoxia +Sm11 N=5. Statistical t test. *:Pval<0.05; **:Pval<0.01; ***:Pval. Data are presented as Mean±SEM. D: Histogram showing that Sm5 and Sm11 promote the acquisition of myelinating OL stage (Gstπ+ cells) following neonatal chronic hypoxia in the cortex at P19. It shows the quantification of Olig2 and Gstπ positive cells. N≥4; Data are presented as Mean±SEM. *p<0.05; **p<0.01; ***p<0.001, unpaired t-test statistical tests.

FIG. 6 is a combination of two histograms showing that Sm5 and Sm11 promote oligodendroglial regeneration in a mouse model of preterm brain injury. A: Quantification of EdU+ cell density (i.e. proliferative cells) in dorsal SVZ at P13 performed through automatic quantification with QPath. B: Quantification of Olig2/EdU cells quantification showing that Sm5 and Sm11 increase the ratio of proliferative OPCs (Olig2+EdU+ cells) in the dorsal SVZ at P13. N≥4; Data are presented as Mean±SEM. *p<0.05; **p<0.01; ***p<0.001, unpaired t-test statistical tests.

### DETAILED DESCRIPTION

In the present invention, the following terms have the following meanings:

“About”, preceding a figure encompasses plus or minus 10%, or less, of the value of said figure. It is to be understood that the value to which the term “about” refers is itself also specifically, and preferably, disclosed.

“Blood-brain barrier”: refers to the selective permeable membrane that regulates the passage of molecules into the extracellular fluid of the central nervous system.

“Mammal” refers to any mammal, including humans, non-human primates, domestic and farm animals, and zoo, sports, or pet animals, such as dogs, cats, cattle, horses, sheep, pigs, goats, rabbits, monkeys, etc. Preferably, the mammal is human.

“Myelin pathologies”: refers to any disease or pathology of the nervous system in which the myelin sheath of neurons is damaged. This term includes demyelinating diseases in which there is a pathological loss of myelin, as well as dysmyelinating diseases, in which the myelin is abnormal and degenerates.

“Salt”: refers to a chemical compound consisting of an ionic assembly of positively charged cations and negatively charged anions, which results in a compound with no net electric charge. This term refers to acid or base addition salts of said compound. The acid addition salts are formed with pharmaceutically acceptable organic or inorganic acids; the base addition salts are formed when an acid proton present in the compound is either replaced by a metal ion or coordinated with a pharmaceutically acceptable organic or inorganic base. In one embodiment, the acid addition salt is selected from the group consisting of acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. In one embodiment, the base addition salt is selected from the group consisting of aluminium, arginine, benzathine, calcium, choline, diethylamine, 2-(diethylamino) ethanol, diolamine, ethanolamine, glycine, 4-(2-hydroxyethyl)-morpholine, lysine, magnesium, meglumine, morpholine, olamine, potassium, sodium, tromethamine and zinc salts. In one embodiment, the salt is a pharmaceutically acceptable salt.

“Small organic molecules” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da. Preferred small organic molecules range in size up to about 5000 g/mol, more preferably up to 2000 g/mol, and most preferably up to about 1000 g/mol.

“Subject” refers to a warm-blooded animal, preferably a mammal, and more preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease.

“Therapeutically effective amount” refers to the level or amount of a small organic molecule as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Treating” or “treatment”: refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a condition or disorder if, after receiving a therapeutic amount of a therapeutic agent according to the methods of the present invention, the patient shows observable and/or measurable reduction in or absence of one or more of the following: reduction in the number of pathogenic cells; reduction in the percent of total cells that are pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.

The present invention relates to a small organic molecule for use in treating or preventing a myelin pathology in a subject in need thereof.

In one embodiment, the small organic molecule according to the present invention targets genes involved in the calcium signaling pathway.

As used herein, calcium signaling refers to the use of calcium ions (Ca²⁺) to communicate and drive intracellular processes often as a step-in signal transduction.

Examples of genes involved in the calcium signaling pathway include, without being limited to, Cacna1a, Cacna1e, Ednrb, Egfr, F2r, Fgfr1, Gna11, Gnas, Itpr2, Ntrk2, Ntrk3, P2rx4, Pde1b, Pdgfra, Ryr2, Ryr3, Camk1, Grm5 and Atp2b3.

In one embodiment, the small organic molecule has a size up to about 5000 g/mol, more preferably up to 2000 g/mol, and most preferably up to about 1000 g/mol.

In one embodiment, the small organic molecule according to the present invention crosses the blood-brain barrier, either directly or indirectly.

In one embodiment, the small organic molecule according to the present invention is able to cross the blood-brain barrier if said small organic molecule is modified to facilitate transport across the blood-brain barrier.

Examples of modifications to facilitate transport across the blood brain barrier include, without limitation, modifying the small organic molecule to allow it to use endogenous carrier-mediated blood-brain barrier transporters such as the glucose transporter type 1 (GLUT1), the large neutral amino-acid transporter type 1 (LAT1), the cationic amino-acid transporter type 1 (CAT1), the monocarboxylic acid transporter type 1 (MCT1), and the equilibrative nucleoside transporter 1 (ENT1); modifying the small organic molecule to increase its lipid solubility by adding lipid groups; covalently coupling the small organic molecule to a blood-brain barrier transportable peptide vector such as cationized albumin, insulin, or transferrin; encapsulating the small organic molecule in lipid- and polymer-based nanoparticles (NPs).

In one embodiment, the small organic molecule according to the present invention is able to cross the blood-brain barrier if said small organic molecule can directly cross the blood-brain barrier. Examples of small organic molecules capable of directly crossing the blood-brain barrier are provided hereinbelow.

In one embodiment, the small organic molecule according to the present invention is selected from the group comprising or consisting of meticrane, heptaminol, melatonin, naringenin, dyclonine, ginkgolide A, levonorgestrel, medrysone, thioperamide, trihexyphenidyl, folinic acid, and salts thereof, and any combination thereof.

In one embodiment, the small organic molecule according to the present invention is selected from the group comprising or consisting of meticrane, heptaminol, dyclonine, ginkgolide A, levonorgestrel, folinic acid, and salts thereof, and any combination thereof.

In one embodiment, the small organic molecule according to the present invention is selected from the group comprising or consisting of dyclonine, folinic acid, salts thereof, and any combination thereof.

In one embodiment, the small organic molecule according to the present invention is dyclonine, or a salt thereof, preferably said small organic molecule is dyclonine hydrochloride. The formula of dyclonine hydrochloride is provided hereinbelow:

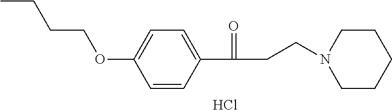 [SMILES: CCCCOc1ccc(C(=O)CCN2CCCCC2)cc1 ; Cl]

Dyclonine hydrochloride (CAS number 536-43-6) is commercially available through the websites of chemical product suppliers.

In one embodiment, the small organic molecule according to the present invention is folinic acid, or a salt thereof, preferably said small organic molecule is calcium folinate. The formula of calcium folinate is provided hereinbelow:

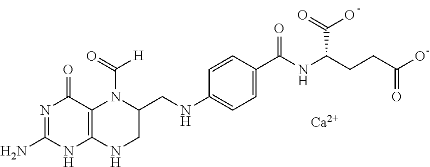 [SMILES: Nc1nc(=O)c2c([nH]1)NCC(CNc1ccc(C(=O)N[C@@H](CCC(=O)O)C(=O)O)cc1)N2C=O ; [Ca+2]]

Calcium folinate (CAS number 1492-18-8) is commercially available through the websites of chemical product suppliers.

In one embodiment, the small organic molecule is heptaminol, or a salt thereof. The formula of heptaminol is provided hereinbelow:

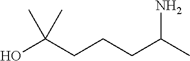

Heptaminol (CAS number 543-15-7) is commercially available through the websites of chemical product suppliers.

In one embodiment, the small organic molecule is meticrane, or a salt thereof. The formula of meticrane is provided hereinbelow:

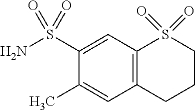 [SMILES: Cc1cc2c(cc1S(N)(=O)=O)S(=O)(=O)CCC2]

Meticrane (CAS number 1084-65-7) is commercially available through the websites of chemical product suppliers.

In one embodiment, the small organic molecule is ginkgolide A, or a salt thereof. The formula of ginkgolide A is provided hereinbelow:

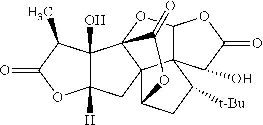

Ginkgolide A (CAS number 15291-75-5) is commercially available through the websites of chemical product suppliers.

In one embodiment, the small organic molecule is levonorgestrel, or a salt thereof. The formula of levonorgestrel is provided hereinbelow:

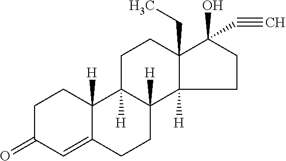

Levonorgestrel (CAS number 797-63-7) is commercially available through the websites of chemical product suppliers.

In one embodiment, the myelin pathology is multiple sclerosis.

As used herein, multiple sclerosis is an immune-mediated disease characterized by gradual decline and finally permanent disabling of motor neurons and sensory functions due to chronic inflammatory demyelination of oligodendrocytes. Clinical manifestations include visual loss, extra-ocular movement disorders, paresthesias, loss of sensation, weakness, dysarthria, spasticity, ataxia, and bladder dysfunction.

Four types of MS have been identified: clinically isolated syndrome (CIS), relapsing-remitting MS (RRMS), primary progressive MS (PPMS), and secondary progressive MS (SPMS). The National Multiple Sclerosis society has provided definitions of these types of MS, that are given hereinbelow.

CIS is a first episode of neurologic symptoms caused by inflammation and demyelination in the central nervous system. The episode, which by definition must last for at least 24 hours, is characteristic of MS but does not yet meet the criteria for a diagnosis of MS. However, it is possible to diagnose MS in a person with CIS who also has specific findings on brain MRI that provide evidence of an earlier episode of damage in a different location and indicate active inflammation in a region other than the one causing the current symptoms.

RRMS is characterized by clearly defined attacks of new or increasing neurologic symptoms. These attacks, also called relapses or exacerbations, are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

SPMS follows an initial relapsing-remitting course. Some people who are diagnosed with RRMS will eventually transition to a secondary progressive course in which there is a progressive worsening of neurologic function (accumulation of disability) over time. SPMS can be further characterized as either active (with relapses and/or evidence of new MRI activity during a specified period of time) or not active, as well as with progression (evidence of disability accumulation over time, with or without relapses or new MRI activity) or without progression.

PPMS is characterized by worsening neurologic function (accumulation of disability) from the onset of symptoms, without early relapses or remissions. PPMS can be further characterized as either active (with an occasional relapse and/or evidence of new MRI activity over a specified period of time) or not active, as well as with progression (evidence of disability accumulation over time, with or without relapse or new MRI activity) or without progression.

In one embodiment, the myelin pathology is a subtype of MS selected from the group consisting of CIS, RRMS, PPMS, and SPMS. In one embodiment, the myelin pathology is the CIS subtype. In one embodiment, the myelin pathology is the RRMS subtype. In one embodiment, the myelin pathology is the PPMS subtype. In one embodiment, the myelin pathology is the SPMS subtype.

In one embodiment, the myelin pathology is associated with or caused by a preterm birth (PTB). In one embodiment, the myelin pathology is associated with or caused by perinatal hypoxia.

As used herein, PTB refers to babies born alive before 37 weeks of pregnancy are completed. There are sub-categories of preterm birth, based on gestational age: i) extremely preterm (less than 28 weeks), ii) very preterm (28 to 32 weeks) and iii) moderate to late preterm (32 to 37 weeks). PTB injury manifests as hypomyelination, interneuron deficit, abnormal connectivity, and synaptopathy.

In one embodiment, the myelin pathology is associated with an extremely preterm birth. In one embodiment, the myelin pathology is associated with a very preterm birth. In one embodiment, the myelin pathology is associated with a moderate to late preterm birth.

In one embodiment, the myelin pathology is not cerebral folate deficiency (CFD). As used herein, CFD is a neurological syndrome in which development is usually normal in the first year of life, but at approximately 2 years of age, affected children start to lose mental and motor skills (psychomotor regression). In one embodiment, the myelin pathology is not associated with folate deficiency, such as CFD. In one embodiment, the myelin pathology is not associated with folate abnormalities.

The present invention further relates to a composition comprising, consisting essentially of or consisting of a small organic molecule as defined hereinabove for use in treating or preventing a myelin pathology in a subject in need thereof.

As used herein, “consisting essentially of”, with reference to a composition, means that the small organic molecule is the only therapeutic agent or agent with a biologic activity within said composition.

The present invention further relates to a pharmaceutical composition comprising, consisting essentially of or consisting of a small organic molecule as defined hereinabove and at least one pharmaceutically acceptable excipient for use in treating or preventing a myelin pathology in a subject in need thereof.

Within the meaning of the invention, the expression “pharmaceutical composition” refers to a composition comprising an active principle in association with a pharmaceutically acceptable vehicle or excipient. A pharmaceutical composition is for therapeutic use, and relates to health.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

The present invention further relates to a medicament comprising, consisting essentially of or consisting of a small organic molecule as defined hereinabove for use in treating or preventing a myelin pathology in a subject in need thereof.

The present invention further relates to the use of a small organic molecule as defined hereinabove in the manufacture of a medicament for treating or preventing a myelin pathology in a subject in need thereof.

The present invention further relates to a method for treating or preventing a myelin pathology in a subject in need thereof, comprising administering to the subject a small organic molecule, a composition, a pharmaceutical composition or a medicament as described hereinabove.

In one embodiment, the small organic molecule according to the present invention induces remyelination. Thus, the present invention further relates to a method for inducing remyelination in a subject in need thereof, comprising administering to the subject a small organic molecule, a composition, a pharmaceutical composition or a medicament as described hereinabove. The present invention further relates to a small organic molecule, a composition, a pharmaceutical composition or a medicament as described hereinabove for use in promoting remyelination in a subject in need thereof.

In one embodiment, the small organic molecule according to the present invention promotes oligodendrogenesis. Thus, the present invention further relates to a method for promoting oligodendrogenesis in a subject in need thereof, comprising administering to the subject a small organic molecule, a composition, a pharmaceutical composition or a medicament as described hereinabove. The present invention further relates to a small organic molecule, a composition, a pharmaceutical composition or a medicament as described hereinabove for use in promoting oligodendrogenesis in a subject in need thereof.

In one embodiment, the small organic molecule according to the present invention promotes differentiation of oligodendrocyte progenitor cells (OPC) into oligodendrocytes. Thus, the present invention further relates to a method for promoting differentiation of OPC into oligodendrocytes in a subject in need thereof, comprising administering to the subject a small organic molecule, a composition, a pharmaceutical composition or a medicament as described hereinabove. The present invention further relates to a small organic molecule, a composition, a pharmaceutical composition or a medicament as described hereinabove for use in promoting differentiation of OPC into oligodendrocytes in a subject in need thereof.

In one embodiment, the small organic molecule according to the present invention promotes oligodendroglia proliferation. Thus, the present invention further relates to a method for promoting oligodendroglia proliferation in a subject in need thereof, comprising administering to the subject a small organic molecule, a composition, a pharmaceutical composition or a medicament as described hereinabove. The present invention further relates to a small organic molecule, a composition, a pharmaceutical composition or a medicament as described hereinabove for use in promoting oligodendroglia proliferation in a subject in need thereof.

In one embodiment, the small organic molecule according to the present invention promotes OPC proliferation. Thus, the present invention further relates to a method for promoting OPC proliferation in a subject in need thereof, comprising administering to the subject a small organic molecule, a composition, a pharmaceutical composition or a medicament as described hereinabove. The present invention further relates to a small organic molecule, a composition, a pharmaceutical composition or a medicament as described hereinabove for use in promoting OPC proliferation in a subject in need thereof.

In one embodiment, the small organic molecule, the composition, the pharmaceutical composition or the medicament according to the present invention is to be administered orally, by injection, topically, nasally, by inhalation, buccally, rectally, intratracheally, by endoscopy, transmucosally, or by percutaneous administration. The term injection used herein includes subcutaneous, intravenous (IV), intramuscular, intra-articular, intra-synovial, intrasternal, intrathecal, intrahepatic, intralesional, perispinal, intracerebral, intraventricular and intracranial injection or infusion techniques.

In one embodiment, the small organic molecule, the composition, the pharmaceutical composition or the medicament according to the present invention is to be administered orally, nasally or by injection, preferably nasally or orally.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of forms suitable for oral administration include, but are not limited to, tablets (including sustained-release tablets), hard capsules, powders, pills (including sugar-coated pills), capsules (including soft gelatin capsules), oral suspensions, oral solutions, and other similar forms.

Examples of forms suitable for nasal administration include, but are not limited to, sprays, nasal drops, nasal ointment and nasal spray solutions.

In one embodiment, the small organic molecule, the composition, the pharmaceutical composition or the medicament according to the present invention is to be administered in a therapeutically effective amount.

It will be understood that the dose of the small organic molecule according to the present invention will be decided by the attending physician within the scope of sound medical judgment. The specific dose for any particular subject will depend upon a variety of factors including the symptom being treated and the severity of the symptom; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well known within the skill of the art to start doses of the compound at levels lower than those required to achieve the desired therapeutic effect and to gradually increase the dosage until the desired effect is achieved.

In one embodiment, the small organic molecule according to the present invention is to be administered at a dose ranging from about 0.01 to about 100 μg per gram per intake (i.e. from about 0.01 to about 100 mg per kg per intake), preferably ranging from about 0.1 to about 10 μg per gram per intake (i.e. from about 0.1 to about 10 mg per kg per intake), more preferably ranging from about 0.5 to about 5 μg per gram per intake (i.e. from about 0.5 to about 5 mg per kg per intake). In one embodiment, the small organic molecule according to the present invention is to be administered at a dose of about 0.5 μg per gram per intake. In one embodiment, the small organic molecule according to the present invention is to be administered at a dose of about 5 μg per gram per intake.

In one embodiment, the dyclonine, preferably the dyclonine hydrochloride, is to be administered at a dose of about 5 μg per gram per intake. In one embodiment, the folinic acid, preferably the calcium folinate, is to be administered at a dose of about 0.5 μg per gram per intake.

In one embodiment, the small organic molecule, the composition, the pharmaceutical composition or the medicament according to the present invention is to be administered once.

In one embodiment, the small organic molecule, the composition, the pharmaceutical composition or the medicament according to the present invention is to be administered several times.

In one embodiment, the small organic molecule, the composition, the pharmaceutical composition or the medicament according to the present invention is to be administered once a day (i.e. daily), every three days, every four days, every five days, every six days, every seven days, every eight days, every nine days, every ten days, every eleven days, every twelve days, every thirteen days, every fourteen days, every fifteen days, once a month, twice a month, once a week, twice a week, at least once a day, twice, or three times a day over a period determined by the skilled man in the art such as, for example, for at least a week, at least a month, for at least two months, at least a year, or more as needed for the rest of the subject's life.

In one embodiment, the small organic molecule, the composition, the pharmaceutical composition or the medicament according to the present invention is to be administered daily, preferably until complete healing of the subject.

In one embodiment, the subject is a male. In one embodiment, the subject is a female. In one embodiment, the subject is an adult, i.e. 18 years old or older. In one embodiment, the subject is a child, i.e. younger than 18 years old. In one embodiment, the subject is a fetus. In one embodiment, the subject is a newborn.

In one embodiment, the subject is affected by, preferably diagnosed with a myelin pathology. Examples of myelin pathologies are provided hereinabove.

In one embodiment, the subject is not affected by folate deficiency, such as CFD. In one embodiment, the subject is not affected with folate abnormalities.

In one embodiment, the subject is affected by or diagnosed with multiple sclerosis.

In one embodiment, the subject is affected by demyelination. In one embodiment, the subject is affected by dysmyelination.

In one embodiment, the subject is treated if said subject presents with signs of remyelination after administration of a small organic molecule, composition, pharmaceutical composition or medicament according to the present invention.

In one embodiment, the subject is treated if said subject presents with signs of reduction of demyelination after administration of a small organic molecule, composition, pharmaceutical composition or medicament according to the present invention.

Means for assessing the level of myelination are well-known by the skilled artisan in the art and include, for example, MRI analyses.

In one embodiment, the subject is considered treated there is a reduction in at least one symptom of the myelin pathology after administration of the small organic molecule, composition, pharmaceutical composition or medicament according to the present invention.

In one embodiment, the subject is considered treated if said subject has an improved lifespan and/or healthspan after administration of the small organic molecule, composition, pharmaceutical composition or medicament according to the present invention.

In one embodiment, the small organic molecule is the only one therapeutic agent to treat or prevent the myelin pathology. Thus, in one embodiment, the small organic molecule is to be used as a monotherapy.

In one embodiment, the small organic molecule is to be administered in combination with another therapeutic agent to treat or prevent the myelin pathology. In one embodiment, the other therapeutic agent is an anti-inflammatory agent.

Examples of anti-inflammatory agents include, without limitation, teriflunomide (CAS number 163451-81-8), fingolimod (CAS number 162359-55-9) and dimethyl fumarate (CAS number 624-49-7). Formulas of these molecules are provided hereinbelow:

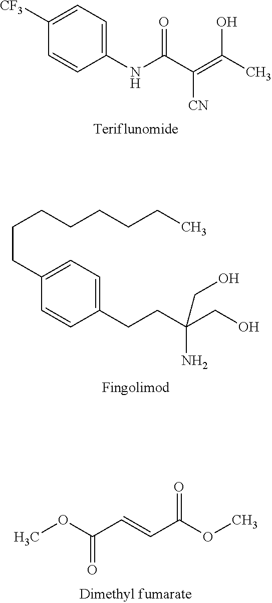

In one embodiment, the small organic molecule, preferably calcium folinate or dyclonine hydrochloride, is to be administered in combination with an anti-inflammatory agent selected from the group comprising or consisting of teriflunomide, fingolimod and dimethyl fumarate.

In one embodiment, the small organic molecule is to be administered to a subject who has received, is receiving or will receive an anti-inflammatory agent as described hereinabove.

The present invention further relates to a kit of parts comprising a small organic molecule and an anti-inflammatory agent as described hereinabove for use in treating or preventing a myelin pathology in a subject in need thereof.

In one embodiment, the other therapeutic agent is another molecule having a pro-myelinating effect. As used herein, a molecule having a pro-myelinating effect is a molecule that can induce or promote myelination of neurons.

In one embodiment, the small organic molecule is to be administered in combination with another molecule having a pro-myelinating effect.

In one embodiment, the small organic molecule according to the present invention is to be administered in combination with another small organic molecule according to the present invention. Thus, in one embodiment, a combination of two, three or more small organic molecules according to the present invention is to be administered to the subject.

Examples of such combinations include the combination of folinic acid, preferably calcium folinate, with at least one of the compounds selected in the group comprising or consisting of meticrane, heptaminol, melatonin, naringenin, dyclonine, ginkgolide A, levonorgestrel, medrysone, thioperamide, trihexyphenidyl, and salts thereof.

Examples of such combination include the combination of dyclonine, preferably dyclonine hydrochloride, with at least one of the compounds selected in the group comprising or consisting of meticrane, heptaminol, melatonin, naringenin, dyclonine, ginkgolide A, levonorgestrel, medrysone, thioperamide, trihexyphenidyl, folinic acid and salts thereof.

Examples of such combination include the combination of calcium folinate with dyclonine hydrochloride.

The present invention further relates to a kit of parts comprising two, three or more small organic molecules as described hereinabove for use in treating or preventing a myelin pathology in a subject in need thereof.

### EXAMPLES

The present invention is further illustrated by the following examples.

### Materials and Methods

### Animals

We used PLPGFP and Swiss mice (Janvier lab). Animals of either sex were included in the study. All animal studies were conducted following protocols approved by local ethical committees and French regulatory authorities.

### Neonatal Neural Stem/Progenitor Cells (NSCs) Cultures

NSC medium was prepared using up to 50 ml DMEM/F12 (Gibco), containing 666 μl glucose at 45% (Sigma), 0.5 ml penicillin/streptomycin (Sigma), 250 μl HEPES buffer at 1M (Gibco), 500 μl mL N2 supplement (Gibco), 1 mL B27 supplement (Gibco) and with growth factors Insulin 20 μg/ml (Sigma), EGF at 20 ng/ml (Peprotech), FGF 10 ng/ml (Peprotech). Brains were collected from Swiss mice at P0 and washed 3 times in PBS 1× (Invitrogen) with 1% of penicillin/streptomycin (Sigma). SVZ was dissected, transferred in fresh NSC medium and dissociated with pipette. Cells were amplified in a 25 ml flask (1 million of cells for 5 mL of medium), and maintained in a humidified atmosphere at 37° C. and 5% of CO2. Two-three days after, cells have grown as floating neurospheres and were collected by centrifugation at 500 g for 5 mn, dissociated before adding proliferating NSC medium. After 2 passages, 30.000 cells were plated in 24 well plates adherent on coverslips, beforehand coated with poly-ornithine (Merck) diluted in 4 volumes of H2O and washed 3 times. Drugs were added at different ranges of concentration (250 nM, 500 nM and 750 nM) and their respective vehicles (DMSO, or PBS). Medium (including drugs or vehicle) was changed each 2 days. After 4 days with proliferation factors, a differentiation medium without growth factors was added (with drugs or vehicle) for two days. Cells were washed once in PBS, fixed in 4% PBS-paraformaldehyde (thermofischer) for 10 minutes, and washed 3 times in PBS-1×.

### Oligodendrocyte Precursor Cells Sorting and Cultures

Brains were collected from PLPGFP and P4 Swiss mice at P0 and washed 3 times in PBS 1× (Invitrogen) with 1% of penicillin/streptomycin (Sigma). Cortex and corpus callosum were dissected and dissociated using neural tissue dissociation kit (T) (Miltenyi biotec) and with dissociator (gentleMACS Octo Dissociator, Miltenyi Biotec). OPC magnetic cell sorting was performed using anti-PDGFRα-coupled-beads (CD140a-PDGFRα MicroBead Kit, Miltenyi biotec) and MultiMACS Cell24 Separator Plus (Miltenyi biotec). Cells were collected in PBS and amplified for 2 days in a proliferating medium (with growth factors IGF, EGF, FGF, and PDGFα). Then, 40.000 cells were plated in 24 well plates adherent on coverslips, beforehand coated with poly-ornithine (Merck) diluted in 4 volumes of H2O and washed 3 times. Successively, for 2 days of proliferation and differentiation, medium was added with drugs at 250 nM, 500 nM and 750 nM, and their respective vehicles (DMSO, PBS). Cells were fixed in 4% PBS-paraformaldehyde (thermo fisher) for 10 minutes and washed 3 times in PBS-1×.

### Cerebellar Organotypic Cultures

We prepared cerebellar organotypic cultures from newborn (P0) mice. Cerebellar parasagittal slices (350 μm thick) were cut on a McIlwain tissue chopper and transferred onto 30 mm diameter Millipore culture inserts with 0.4 mm pores (Millicell, Millipore). Slices were maintained in incubators at 37° C., under a humidified atmosphere containing 5% CO2 in six-well-plates containing 1 mL of slice culture media. This medium includes basal medium eagle (Invitrogen), 25% complete HBSS, 27 mM D-glucose, 100 U/mL penicillin/streptomycin, 1 mM glutamine (Sigma) and 5% horse serum (New Zealand origin, heat inactivated; Invitrogen). Medium was renewed every 2 days until 7 DIV, reaching the appropriate culture periods to study OPC differentiation and myelination. At this time point, we added small molecules each day for 3 DIV (Sm1, Sm2, Sm5, Sm11 at 750 nM), and their respective vehicles (DMSO at 750 nM or PBS). Slices were collected and fixed for 1 h at RT in PBS-4% paraformaldehyde, washed 3 times in PBS, and incubated 20 min at 4° C. in Clark's solution (95% ethanol/5% acetic acid), then washed 3 times in PBS 1×.

### Intracerebral Injections of Lysolecithin Induced Demyelination in the Adult Mice

We weighted and anesthetized 4 months old RjOrl: SWISS mice (Janvier) with isoflurane (induced at 3%, and maintained at 1.2-1.6%). We induced focal demyelinating lesions by injecting 0.5 μl of a solution of 1% lysolecithin (LPC, Sigma in 0.9% NaCl solution) into the corpus callosum. To do so, a glass-capillary connected to a 10 μl Hamilton syringe was fixed and oriented through stereotaxic apparatus (coordinates: 1 mm lateral, 1.3 mm rostral to Bregma, 1.7 mm deep to brain surface). Animals were left to recover for a few hours in a warm chamber. Control and drugs sm5 and sm11 (sm5 at 5 μg per g and sm11 at 035 μg per g) were administered daily in 5%-glucose drinking water and their consumption was measured. Mice was perfused at 2% cold paraformaldehyde (thermos fisher) at 7 DPI.

### Neonatal Hypoxia

Mice aged P1 were placed in a hypoxic rearing chamber maintaining at 9.5-10.5% O2 concentration by displacement with N2. Hypoxia began at the postnatal day 3 (P3) for 8 days until P11. A separate group was maintained in a normal atmosphere (normoxic group). Drugs were administered by intranasal administration with sm5 at 5 μg per g and sm11 at 0.5 μg per g. Mucus was first permeabilized by the use of type IV hyaluronidase, then 10 μl of drugs (Sigma) was administered 3 times, 1 time a day from P11 to P13 (starting at the end of the hypoxic period, then every 24 hours) in sterile PBS (control). For analysis of proliferation, mice were injected with EdU (Sigma) in order to label cells in an actively cycling state in S-phase, then perfused 1 h later at P13. For analysis of OL maturation, mice were perfused at P19. All perfusions were performed with Ringer, followed by ice cold solution of 4% paraformaldehyde (Thermo Fisher). Mice were sacrificed at P13 or P19 by an intraperitoneal overdose of pentobarbital followed by perfusion with Ringer's lactate solution and 4% paraformaldehyde (PFA; Sigma) dissolved in 0.1M phosphate buffer (PB; pH 7.4). Brains were removed and post-fixed for 24 hours at 4° C. in 4% PFA and sectioned in 50 μm thick coronal serial sections, free floating sections.

### Immunochemistry

For coated-coverslip, blocking was performed in a moist chamber for 30 mn at room temperature (RT) in the blocking buffer (0.05% Triton X-100/10% normal goat serum/PBS). Then, coverslip were incubated for 30 mn with the following primary antibodies at RT. Rat anti-PDGFRα (1:250, BD Biosciences); Mouse anti-CNPase (mlgG1, 1:250; Millipore); Rabbit anti-Btub (1:1000; BABCO); Chicken anti-GFAP (1:500, Avelabs), Mouse anti-MBP (mlgG1, 1:150; Abcam); Rabbit anti-Sox10 (1:1000; Proteintech). Following 3 washing in 0.05% Triton X-100/PBS, coverslips were incubated 30 min at RT in dark with appropriate secondary antibodies conjugated with Alexa Fluor 488, 594 and 647 (1:1000; Molecular Probes or Thermo Fisher) and Dapi (1:5000 from working solution at 5 mg/ml; Life Technologies; D1306). Coverslips were washed 3 times in 0.05% Triton X-100/PBS and mounted with Fluoromount-G (SouthernBiotech).

For ex-vivo cerebellar slices, slices were incubated 20 min at 4° C. in Clark's solution (95% ethanol/5% acetic acid), then washed 3 times in PBS 1×. Then, sections were incubated for 1 hour in a blocking buffer (0.2% Triton X-100/4% bovine serum albumin/4% donkey serum/PBS). Slices were incubated for 2 hours with the following primary antibodies at RT: Goat anti-Sox10 (1:100, R&D Systems); Rabbit anti-Calbindin (1:1000; Swant); Rat anti-MBP (1:200; Abcam); Mouse anti-CC1 (IgG2b; 1:100, Calbiochem). Then slices were washed 3 times in PBS/0.1% Triton X-100 and incubated in blocking buffer (dark, RT) for 2 hours with appropriate secondary antibodies conjugated with Alexa Fluor 488, 594 and 647 (1:1000; Molecular Probes or Thermo Fisher) and Dapi (1:5000 from working solution at 5 mg/ml; Life Technologies; D1306). Finally, slices were washed 3 times in PBS/0.1% Triton X-100 after incubation and mounted in Fluoromount-G (SouthernBiotech).

For in-vivo cryosections (14-μm thick) of lesions, immunohistochemistry was performed after 30 mn drying at RT in a moist chamber. For myelin staining, ethanol treatment (100%) was performed for 10 mn at RT. Briefly, sections were permeabilized and blocked in a blocking buffer for 1 h (0.05% Triton X-100/10% normal goat serum/PBS). Then, cryosections were incubated overnight with the following primary antibodies at 4° C.: Mouse anti-APC (CC1 IgG2B; 1:100, calbiochem); Rabbit anti-Olig2 (1:500; Millipore); Mouse anti-Olig1 (IgG1; 1:500; NeuroMab); Rat anti-CD68 (1:500, AbD SEROTEC/BioRad); Rabbit anti-IBA1 (1:500, Wako Fujifilm); Mouse anti-MOOG (mlgG1, 1:20; Hybridoma). Then sections were washed 3 times in 0.05% Triton X-100/PBS and incubated 1 hour at RT with appropriate secondary antibodies conjugated with Alexa Fluor 488, 594 and 647 (1:1000; Molecular Probes or Thermo Fisher) and Dapi (1:5000 from working solution at 5 mg/ml; Life Technologies; D1306). Sections were washed 3 times in 0.05% Triton X-100/PBS and mounted with Fluoromount-G (SouthernBiotech).

For in vivo-cryosections (50 μm) of chronic hypoxia, antigen retrieval was performed for 20 min in a citrate buffer (ph 6.0) at 80° C., then cooled for 20 min at room temperature and washed in 0.1M PB. The following antibody were used for immunohistochemical procedures: Mouse anti-APC (CC1 IgG2B; 1:100, calbiochem); Mouse anti-Olig2 (1:1500; Millipore); Rabbit anti-Olig2 (1:400; Millipore); Rabbit anti-GSTpi (1:1000, Enzo). Blocking was performed in a TNB buffer (0.1 M PB; 0.05% Casein; 0.25% Bovine Serum Albumin; 0.25% TopBlock) with 0.4% triton-X (TNB-Tx). Sections were incubated overnight at 4° C. with gentle shaking. Following extensive washing in 0.1 M PB with 0.4% triton-X (PB-Tx), sections were incubated with appropriate secondary antibodies conjugated with Alexa Fluor 488 or 555 (1:500; Jackson or Invitrogen) for 2 hrs at room temperature. Sections were washed and counterstained with Dapi (1:5000 from a working solution at 5 mg/ml; Life Technologies; D1306). Revelation of EdU was done using Click-it™, EdU cell proliferation Kit for imaging, Alexa fluor™ 647 dye (Thermo Fisher scientific).

### Image Acquisition and Analysis

Acquisitions for cryosections and ICC were performed with Zeiss microscope using either apotome system optical sectioning and Axioscan (Zeiss) at ×20 magnification, including deconvolution and Z-stack. Acquisition for cerebellar slices was done using a confocal SP8 X white light laser at ×40 magnification (Leica). Analysis was performed using ZEN (Zeiss) and ImageJ (Fiji) software packages, with macros for myelination and differentiation index ex-vivo. Manual quantifications for in-vivo lesions analysis was performed blindly. Illustrations were done using Illustrator and Adobe Photoshop (Adobe System, Inc).

### Statistics and Representations

Statistical analysis was conducted using Graphpad (Prism) and R Studio version 3.6.1 (R Development Core Team, 2019). For in-vitro experiments, analysis of differences between conditions and control groups was performed by fitting a linear mixed-effects models (LMM) to the percentage data. The condition factor was used as a fixed effect, while the number of experiments (1 to 6) was assigned as a random effect (intercept) to account for correlation of measurements from the same experience. LMM was fitted using restricted maximum-likelihood estimation (REML) from the function Imer in the 1me4 package (Bates, D., Mächler, M., Bolker, B., & Walker, S. (2015). Fitting Linear Mixed-Effects Models Using Ime4. Journal of Statistical Software, 67 (1), 1-48) (v1.1-21). Significance for the main effect of Condition was assessed based on Type II Wald chi-square tests using the function ANOVA in the car package (v3.0-7). Pairwise comparisons between the conditions and control groups were performed by specifying custom contrasts with the emmeans package (v1.4.5). P-values resulting from the comparisons were obtained using Kenward-Roger's approximation for degrees of freedom, and after adjustment with the FDR method to account for the multiplicity of contrasts. For morphological analysis, significance was determined using one-way ANOVA by multiple comparisons followed by pairwise comparisons with Dunnett's post-hoc test. For ex-vivo experiments, unpaired bilateral Wilcoxon Mann Whitney test was performed to evaluate difference between each pair of variables (groups), followed by unpaired Kruskal Wallis. For in-vivo experiments, statistical significance was calculated using unpaired T-test. Statistical significance was set at p or adjusted-p<0.05 (significativity: * Pval<0.05; ** Pval<0.01; *** Pval<0.001). Data are presented as mean±SEM.

### Results

### Pro-Oligodendrogenic Activity of Small Molecules in Neonatal Neural Progenitor Cultures

We select the following 11 drugs to test their in vitro activity in oligodendrogenesis (See Table 1 below).

TABLE 1

| Nomenclature | Molecule |
|---|---|
| Sm1 | Meticrane |
| Sm2 | Heptaminol |
| Sm3 | Melatonin |
| Sm4 | Naringenin |
| Sm5 | Dyclonine hydrochloride |
| Sm6 | Ginkgolide A |
| Sm7 | Levonorgestrel |
| Sm8 | Medrysone |
| Sm9 | Thioperamide |
| Sm10 | Trihexyphenidyl |
| Sm11 | Calcium folinate |

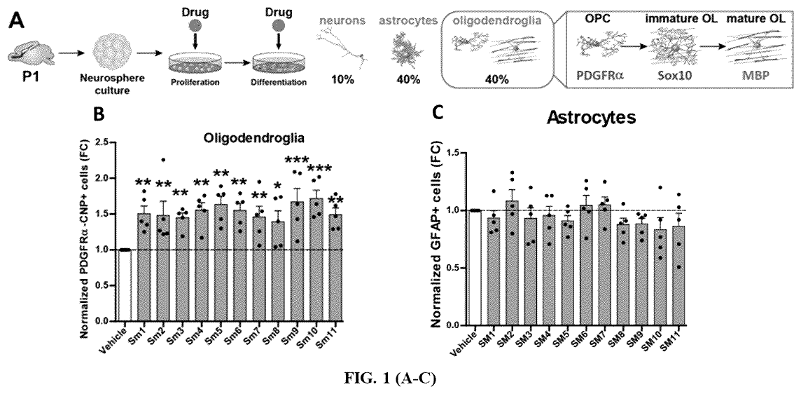

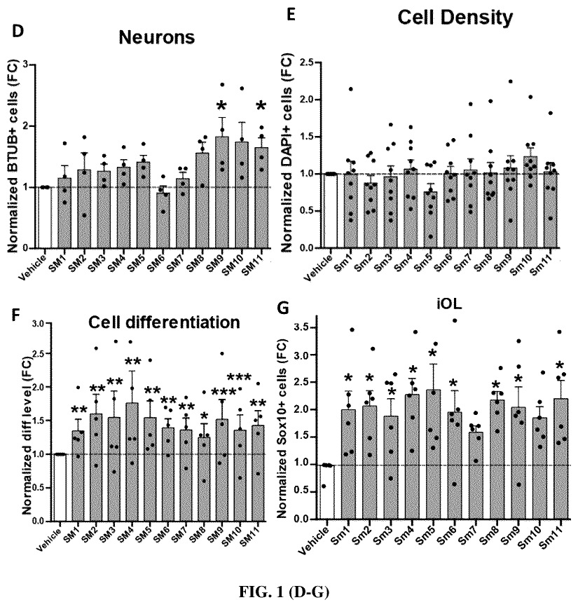

To do so, we first used a culture of mix neural progenitor cells (NPCs) obtained from the neonatal SVZ (postnatal day 1, P1). We optimized the protocol to increase the proportion of oligodendroglia by plating NPCs in the proliferation medium containing EGF, FGF, and PDGF, obtaining ˜40% of oligodendroglial cells, ˜40% of astrocytes and ˜10% of neurons after allowing differentiation by removing the growth factors (FIG. 1A). We supplemented the drugs in both the proliferation and differentiation medium to analyze both their effect in neural cell type specification into the different neural lineages, and assess their ability to foster OPC differentiation. We used the following range of drug concentrations 250 nM, 500 nM, and 750 nM to test for possible drug cytotoxicity. We used standard markers to immunodetect the main neural cell subtypes, neurons (β-III-tubulin), astrocytes (GFAP), and oligodendroglia (combining PDGFRα and CNP). We found a significant effect of all the selected drugs at 750 nM to promote a 1.5-fold increase in oligodendroglial cells (PDGFRα+ and/or CNP+ cells) (FIG. 1B). The increase in the number of oligodendroglial cells promoted by the selected small molecules was not accompanied by significant reductions in the number of neurons and astrocytes, or changes in cell density (FIG. 1C-E), but paralleled by a significant increase in cell differentiation (reduction of markers-negative undifferentiated cells; FIG. 1F), suggesting that the selected drugs foster cell differentiation mainly towards the oligodendroglial fate, with only sm9 and sm11 also showing a positive effect in neurogenesis (FIG. 1D).

To determine drugs effect on the OL lineage, we optimized a protocol of immunodetection to label their different stages: OPCs (PDGFRα+ cells), immature OLs (iOLs, Sox10high cells, some starting to express MBP), and mature OLs (mOLs, Sox10low/MBP+ cells). We found a ˜2-fold increase in iOLs (Sox10high cells) for all the selected drugs (FIG. 1G), with only sm7 and sm10 showing increased tendency not reaching statistical significance, due to the larger experimental variance. Altogether, these results suggest that the selected drugs have a pro-oligodendrogenic effect in NPC differentiation cultures without having a negative impact in the numbers neurons and astrocytes.

### Pro-Oligodendrogenic Activity of Small Molecules in Primary OPC Cultures

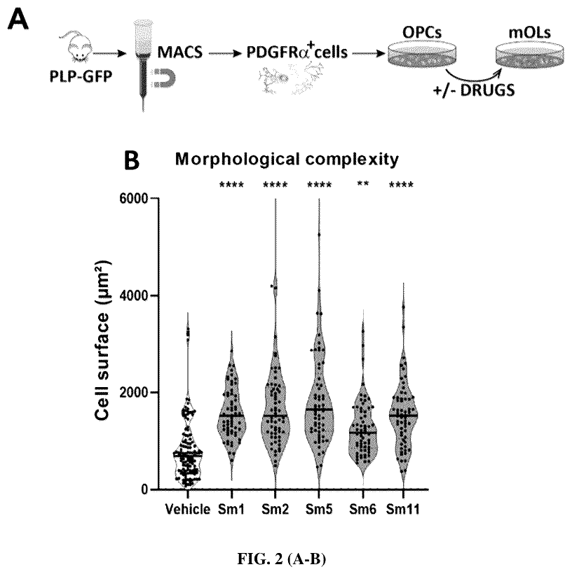

To evaluate cell specific contribution of the drugs directly in OL differentiation and maturation, we used primary OPCs purified by magnetic cell sorting (MACS) from neonatal cortices (P4). We treated OPCs for two days in presence of the five molecules Sm1, Sm2, Sm5, Sm6, Sm11, and assess their level of differentiation/maturation by measuring their morphological complexity (FIG. 2A). Interestingly, all drugs tested strongly increased the morphological complexity of OLs (PLP+ cells), compared to their negative control (vehicle; FIG. 2B). These results demonstrate the capacity of the selected drugs to directly foster OPC differentiation and increase the morphological complexity of differentiating OLs, a trait of their level of maturation.

### Pro-Oligodendrogenic Activity of Small Molecules in Cerebellar Explants

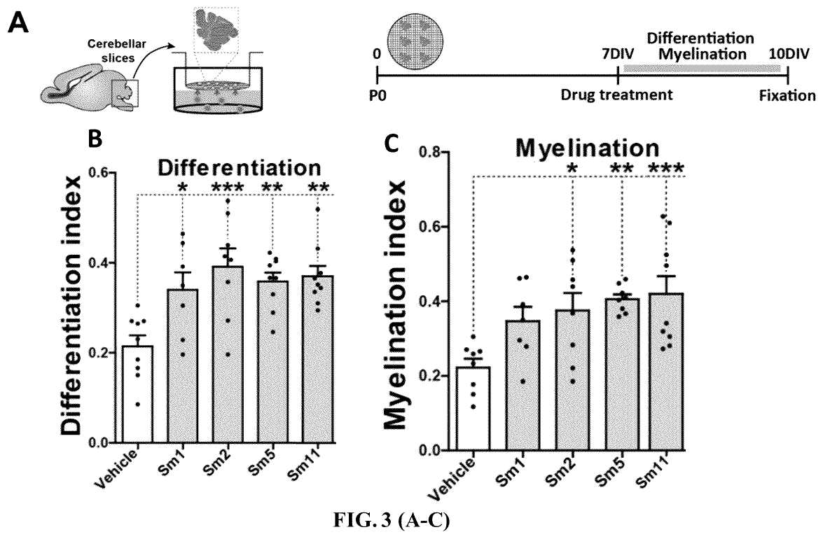

Then, we firstly performed a simpler and faster experimental model using cerebellar slices explants to identify the best drugs having pro-oligodendrogenenic and pro-myelinating activities in this ex vivo model, to be used in the in vivo analyses. Indeed, this model allowed us to better assess later aspects of OL maturation, myelination, and cytotoxicity in a richer cellular system, also containing immune cells (microglia/macrophages) that are known to influence oligodendrogenesis and (re) myelination. In this model, the start of myelination takes place after 7 days in culture (FIG. 3A). Therefore, we analyzed the effect of incubating the cerebellar explants for three days (7-10 days in vitro, DIV) in the presence of the top-4 small molecules having higher pro-oligodendrogenic activity in culture (sm1, sm2, sm5, sm11), and their associated negative controls (vehicle: DMSO or PBS). We then analyzed the effect in terms of OL numbers (CC1+/Sox10+ cells) and myelination (MBP labeling on CaBP+ Purkinje cell axons), by immunodetection of all four markers in the same sections. We calculated the differentiation index as the ratio of Sox10+ cells being CC1+, and the myelination index as the ratio CaBP+ axons being MBP+. Remarkably, all drugs presented an increased differentiation index (CC1+/Sox10+ OLs number) compared to their negative controls (FIG. 3B). Moreover, sm2, sm5, and sm11 induced a robust increase in the myelination process (MBP+/CaBP+), when compared to vehicle (FIG. 3C). Altogether, based on the global effects of these molecules combining our in vitro and ex vivo experiments, we selected sm5 and sm11 as the best candidates to assess their efficiency to promote oligodendrogenesis and (re) myelination in two in vivo mouse models. On the one hand, we used the adult focal de/remyelination induced by intracerebral injection of lysolecithin-lipotoxin (LPC) mimicking de/remyelination of MS, and on the other hand, the neonatal chronic hypoxia model, mimicking PTB hypomyelination.

### Assessing Pro-Oligodendrogenic Activity of Sm5 and Sm11 in a Focal De/Remyelination Mouse Model

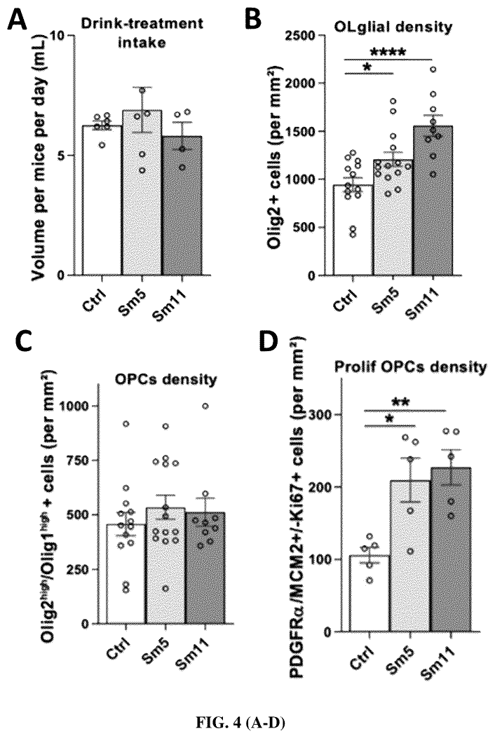

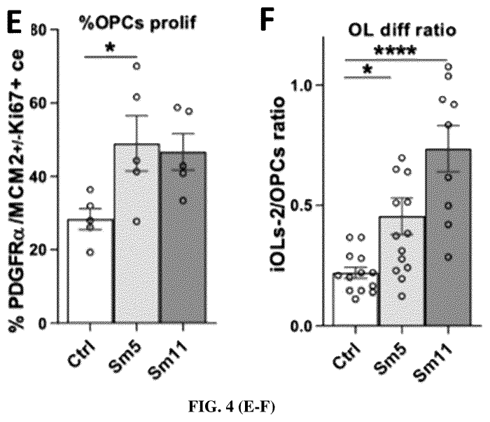

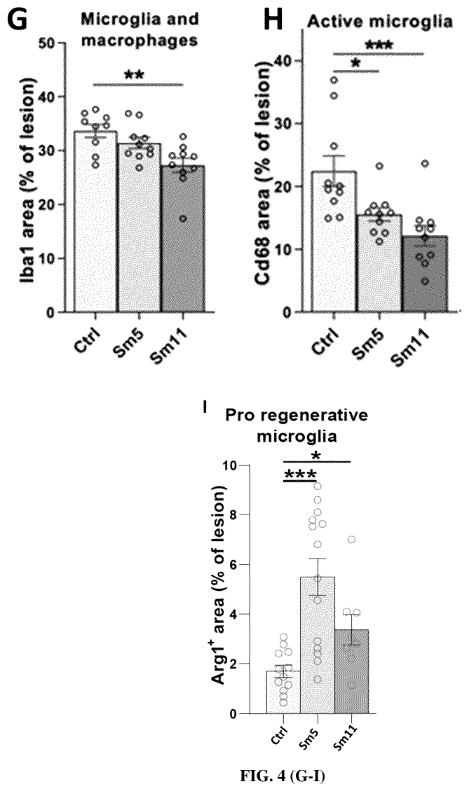

To assess the pro-oligodendrogenic capacity of sm5 and sm11 in vivo in the context of adult remyelination, we used lysolecithin (LPC) to induce the breakdown of lipid-rich myelin membranes and focal demyelination in the corpus callosum of adult (4 months old) mice. We administered drugs in the drinking water daily, and did not find any significant changes in daily oral consumption among groups (FIG. 4A). We analyzed the lesions at 7 days post-lesion (7 dpl), when newly formed OLs have started to remyelinate the lesion in this model. The lesion area was identified by immunodetection of the high cellular density with DAPI staining, reduction of myelin with myelin oligodendrocyte glycoprotein (MOG), and abundance of microglia/macrophages with Iba1 and Cd68. We determined the effect of the drugs to promote oligodendrogenesis at the lesion site by combinatory immunodetection of Olig2, CC1 and Olig1, allowing to distinguish OPCs (Olig2^{high}/CC1⁻/Olig1^{nuclear-cyto} cells) and three stages in OL differentiation: iOL1 (Olig2^{high}/CC1^{high}/Olig1⁻ cells), iOL2 (Olig2^{high}/CC1^{high}/Olig1^{high-cyto} cells), and mOL (Olig2^{low}/CC1^{low}/Olig1^{low} cells). First, we found that sm5 and more strongly sm11 increase the number of oligodendroglial cells in the remyelinating area (respectively 1208±281.1 and 1558±331 Olig2+ cells per mm² compared to the control presenting 942.5±265.7 Olig2+ cells per mm²; FIG. 4B). Quantification of oligodendroglial stages showed that the administration of the drugs did not change the density of OPCs (Olig2^{high}/CC1⁻/Olig1^{nuclear-cyto} cells) in the lesion area (FIG. 4C). In addition, we observed that sm11 increased by ˜2-fold each stage of OL differentiation (iOL1, iOL2 & mOL) while sm5 showed a more moderate effect only significant at the iOL2 stage. Given the increase in OL differentiation by the drugs administration, without diminishing the pool of OPCs in the lesion area, we assessed for the proliferative status of OPCs, using Mcm2 and Ki67 proliferative markers, finding that both drugs promote a ˜2-fold increase in the density of proliferative OPCs (FIG. 4D) and in the proportion of proliferative OPCs (FIG. 4E). Finally, calculation of the differentiation ratio (number of OLs per OPC), showed that whereas for the control was 25% (1 OLs for 4 OPCs), it increased by 2-fold with sm5 (50%, 2 OLs for 4 OPC) and 3-fold with sm11 (85%, 3 OLs for 4 OPCs; FIG. 4F). Altogether, these results obtained in vivo, showing the capacity of sm5 and sm11 to promote OL differentiation are consistent with our results obtained in vitro and ex vivo, thus suggesting that similar results could be obtained for the other selected drugs non tested yet in vivo. Moreover, the increased number of oligodendroglial cells (Olig2+ cells) in the lesion area upon together with the increase in OPC proliferation, indicates that these drugs not only promote OL differentiation but also are able to sustain pool of OPCs by also fostering their cycling status.

To investigate the possible anti-inflammatory and pro-regenerative activity of these drugs, we performed staining for microglia/macrophages (Iba1+ cells), their active phagocytic status (Cd68+ cells), together with myelin (MOG) and cell density (DAPI) to identify the lesion area. Interestingly, surface occupied by Iba1+ microglia/macrophages in the lesion area (high DAPI density area) decreased in response to sm11 (27.28%±4.16% in sm11 versus 33.64%+3.63% in control; FIG. 4G), accompanied by a ˜2-fold reduction in the actively phagocytic microglia/macrophages (Cd68+ cells, 12.13%±5.14% in sm11 versus 22.45%+7.70% in control) (FIG. 4H). In the case of sm5 treatment, there was also a trend to reduce the density of microglia/macrophages, mainly explained by a reduction of their phagocytic population (FIG. 4G-H). Of note, the reduction of phagocytic microglia was paralleled with a corresponding increase in pro-regenerative microglia (Arg1+ cells) (FIG. 4I). Together, these results suggest that sm11, and to a lesser extent sm5, accelerate repair-associated processes not only by promoting OPC differentiation and proliferation but also through acceleration of myelin debris clearance likely by decreasing fraction of microglia with pro-inflammatory phenotype.

In summary, these results demonstrate that oral administration of sm5 and sm11 induces a pro-oligodendrogenic activity in vivo in the context of adult remyelinating brain, and that sm11, and to less extend sm5, also improved lesion repair reducing inflammation and fostering the phagocytic activity of microglia.

Assessing Activity of Sm5 and Sm11 for Oligodendroglial Regeneration in White Matter after Neonatal Hypoxia

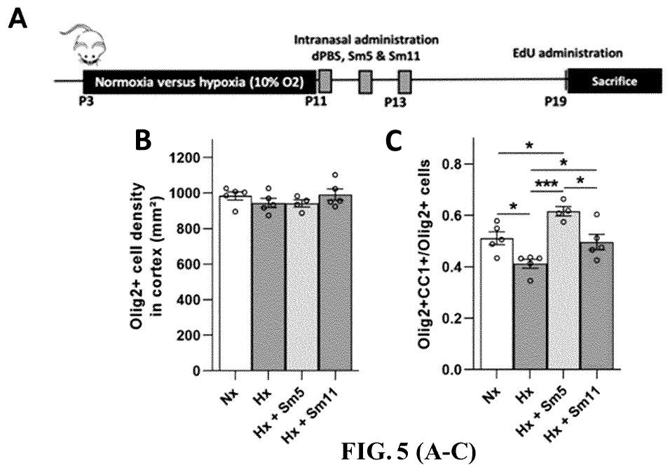

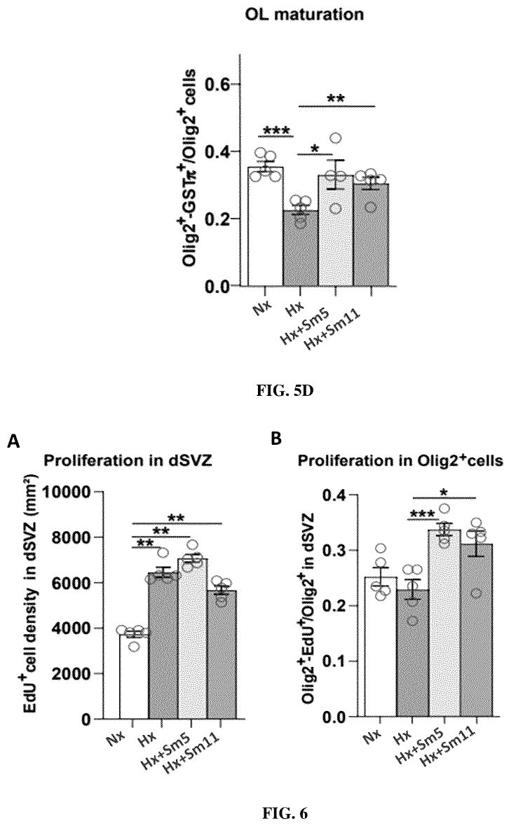

Chronic hypoxia is a well-established model of very preterm birth, that induces a diffuse perinatal brain lesion by housing the pups at 10% oxygen from P3 to P11 (FIG. 5A). This neonatal hypoxia induces a marginal cell death including neuronal and glial cells along with delay into forebrain development including OL maturation that appear to persist in adulthood. Thus, we assessed sm5 and sm11 effects, as pro-oligodendroglial treatment, following hypoxia. We performed an intranasal administration of sm5 or sm11 for three days from P11 (end of hypoxic period) to P13 (Methods). This approach allows a non-invasive administration of drugs that access the brain because of their capacity to cross the blood brain barrier. To examine the impact of Sm5 and Sm11 on OL lineage proliferation, brains were analyzed at P13, corresponding to the end of the treatment period, following an administration of EdU one hour before sacrifice to label cells in S-phase. Quantification of EdU+ cell density within the subventricular zone revealed an overall increase of proliferation following hypoxia, with no marked additive effects of the treatment (FIG. 6A). Analysis of the proportion of Olig2+ cells labeled with EdU revealed a significant increase following both Sm5 and Sm11 (FIG. 6B), suggesting an increase in the proliferation of oligodendroglial committed progenitors. To look at the impact of sm5 and sm11 on OL lineage proliferation and maturation, brains were analyzed within the cortex six days following the last administration (P19). We first saw that the density of oligodendroglial cells (Olig2+ cells, FIG. 5B) was similar between normoxic (i.e. control condition) and hypoxic groups, with no major effect produced by the administration of the drugs (FIG. 5B). We next assessed the impact on OL differentiation by immunodetection of OLs with CC1 antibody and oligodendroglial cells by Olig2. We confirmed that hypoxia significantly decreases OL differentiation, as shown by the decreased ratio of Olig2+ cells expressing CC1 (FIG. 5C). Remarkably, both drug treatments rescued this ratio compared to the hypoxic group (FIG. 5C). Interestingly, sm11 treatment rescued the ratio at a similar level than the normoxic group, whereas sm5 increased OL differentiation beyond the normoxic and sm11 groups (FIG. 5C). We then assessed whether the number of myelinating OLs was also increased by the treatments, using GSTπ as a marker restricted to myelinating OLs. Hypoxia resulted in a preferential reduction of myelinating OLs (GSTπ+ cells) compared to all OLs (CC1+ cells) (FIG. 5D). Interestingly, treatment with both Sm5 and Sm11 resulted in a complete recovery of GSTπ expression (FIG. 5D). These results are in line with our previous in vitro and in vivo results and confirm the therapeutic capacity of sm5 and sm11 to promote oligodendrogenesis in a model of preterm birth brain injury. This also validates the intranasal approach to administer these drugs and their capacity to cross the BBB.

Altogether, our results demonstrate the efficacy of the top two selected drugs to promote oligodendrogenesis and lesion repair in two mouse models mimicking two common myelin pathologies, the preterm brain injury (neonatal hypoxia model) and Multiple Sclerosis (adult focal brain de (re) myelination).

1-15. (canceled)

16. A method for treating or preventing a myelin pathology in a subject in need thereof, comprising administering to the subject:
a small organic molecule that targets the genes involved in the calcium signaling pathway, or
a pharmaceutical composition comprising, consisting of or consisting essentially of said small organic molecule and a pharmaceutically acceptable excipient.

17. The method according to claim 16, wherein said small organic molecule crosses the blood-brain barrier.

18. The method according to claim 16, wherein said small organic molecule is selected from the group comprising or consisting of folinic acid, dyclonine, or salts thereof, and any combination thereof.

19. The method according to claim 16, wherein said small organic molecule is folinic acid or a salt thereof.

20. The method according to claim 19, wherein said small organic molecule is calcium folinate.

21. The method according to claim 16, wherein said small organic molecule is dyclonine or a salt thereof.

22. The method according to claim 21, wherein said small organic molecule is dyclonine hydrochloride.

23. The method according to claim 16, wherein the myelin pathology is not associated with folate deficiency.

24. The method according to claim 16, wherein the myelin pathology is multiple sclerosis.

25. The method according to claim 16, wherein the myelin pathology is associated with preterm birth.

26. The method according to claim 25, wherein the myelin pathology is associated with perinatal hypoxia.

27. The method according to claim 16, wherein said small organic molecule is to be administered orally, by injection, topically, nasally, by inhalation, buccally, rectally, intratracheally, by endoscopy, transmucosally, by percutaneous administration.

28. The method according to claim 27, wherein said small organic molecule is to be administered orally or nasally.

29. The method according to claim 16, wherein said small organic molecule is to be administered between about 0.01 and about 100 μg per gram per intake.

30. The method according to claim 29, wherein said small organic molecule is to be administered between about 0.1 and about 10 μg per gram per intake.

31. The method according to claim 29, wherein said small organic molecule is to be administered between about 0.5 and about 5 μg per gram per intake.

32. The method according to claim 16, wherein said small organic molecule is to be administered daily.

33. The method according to claim 16, wherein said subject has received, is receiving or will receive an anti-inflammatory agent.

34. The method according to claim 33, wherein said anti-inflammatory agent is selected from the group comprising or consisting of teriflunomide, fingolimod and dimethyl fumarate.

35. The method according to claim 16, wherein said subject is not affected by folate deficiency.
